(19) United States
(12) Patent Application Publication
(10) Pub. No.: US 2022/0280945 A1
(43) Pub. Date: Sep. 8, 2022

(54) PACKAGING FOR ASSAYS AND RELATED METHODS

(71) Applicant: Meso Scale Technologies, LLC., Rockville, MD (US)

(72) Inventors: Jacob N. Wohlstadter, Palm Beach Gardens, FL (US); Paul J. Goodwin, Frederick, MD (US); Staci Leffel, Brattleboro, VT (US); Joseph Seppy, North Potomac, MD (US)

(21) Appl. No.: 17/686,900

(22) Filed: Mar. 4, 2022

Related U.S. Application Data
(60) Provisional application No. 63/156,837, filed on Mar. 4, 2021.

(51) Int. Cl. B01L 3/00; G06K 19/06; G01N 35/10; G01N 35/00

(52) U.S. Cl. CPC B01L 3/545; G06K 19/06018; G01N 35/1002; G01N 35/00732; G01N 35/00663; B01L 2300/021; B01L 2200/16

(57) ABSTRACT

The disclosure relates to a package for an assay kit, comprising an insert that provides for convenient, e.g. one step, scanning of reagent and other assay kit information from assay kit reagent containers that are disposed within the insert. The disclosure also describes methods of preparing such an insert and of assembling the assay kit.

### FIELD

The disclosure is directed to an assay kit package, an insert for same, and methods of preparing the insert and for manufacturing, assembling, storing, and reassembling an assay kit.

### BACKGROUND

Heretofore, it was problematic to quickly identify and correctly match assay kit reagents and components for assays for quick and accurate assembly, shipping and reassembly, including for example, where portions of the assay kit may be shipped or stored separately, including at different temperatures and/or at different locations. Such logistics typically require some disassembly of the related packages to identify contents and/or reliance on identifier coding that oftentimes requires extensive cross referencing and/or undue machinations by personnel to ensure proper assembly during manufacture or during reunification. Hence, an improved package and methods which permit the assay kit reagents and components to be quickly and reliably assembled, shipped, and subsequently reassembled by the customer is needed, and which also provide greatly improved operational experiences at the manufacturer and user level.

### SUMMARY

In one practice, the disclosure provides a package for a particular assay kit comprising a box, optionally having assay product information on an outer sidewall; an insert disposed within the outer box, the insert configured to hold a plurality of assay kit reagent containers comprising the same or different reagents, wherein each assay kit reagent container or a subset thereof, has a container bottom on which is located a product code that is readable by a human, a machine, or both and identifies the reagent within that particular container, the insert comprising an upper surface, a bottom surface, and a plurality of through holes extending through the insert from the upper surface to the bottom surface, each of the plurality of through holes shaped to individually hold a respective assay kit reagent container such that the product code located on container bottoms, or subset of container bottoms, comprising the product code is readable through the respective through hole at the bottom surface of the insert.

The disclosure also provides a loaded insert that is filled or partially filled with assay kit reagent containers for a tray or a outer box, wherein the loaded insert comprises an upper surface, a bottom surface, and a plurality of through holes extending from the upper surface of the insert to the bottom surface of the insert; and a plurality of assay kit reagent containers comprising the same or different reagents, each of the plurality of assay kit reagent containers disposed within a respective through hole of the plurality of through holes, each of the plurality of assay kit reagent containers or a subset thereof having a container bottom on which is located a product code identifying the reagent within that particular container, the product code readable by a human or a machine or both through the respective through hole at the bottom surface of the loaded insert.

The disclosure additionally provides a method of preparing a loaded insert for an assay kit comprising providing a plurality of assay kit reagent containers associated with a particular assay kit, the plurality of assay kit reagent containers comprising the same or different reagents, each of the plurality of assay kit reagent containers or a subset thereof having a container bottom on which is located a product code identifying the reagent within that particular container, the product code readable by a human or a machine or both; providing an insert that comprises an upper surface, a bottom surface, and one or more through holes extending from the upper surface of the insert to the bottom surface of the insert; preparing a loaded insert by disposing each of the plurality of assay kit reagent containers into a respective through hole of the plurality of through holes such that the product code on each container bottom or subset comprising the product code is readable through the respective through hole at the bottom surface of the insert; inputting the product code on each container bottom or subset comprising the product code into a database accessible by a computer, the database comprising information about reagents for the particular assay kit; and comparing the product code on each container or subset comprising the product code with the information in the database about the reagents for the particular assay kit to verify that the reagents contained within the plurality of assay kit reagent containers are for the particular single assay kit. In another practice, the location of each container in the loaded insert and its contents are recorded and stored in the database, e.g. this information forms part of the inputting of product code into the database.

An aspect of the disclosure provides a method for storing and retrieving an assay kit comprising (i) providing, to a customer, outer boxes for a particular assay kit, each outer box comprising (a) an outer sidewall on which is an assay kit collection code that is human readable, machine readable or both and is uniquely associated with that particular assay kit, and (b) optionally, one or more inserts disposed within one or more of each outer box, the insert holding a plurality of assay kit reagent containers comprising the same or different reagents; (ii) storing respective outer boxes at storage locations which can be the same location or different locations, including different storage conditions, depending on the reagents contained within the assay kit reagent containers; and (iii) retrieving, when ready to assemble the particular single assay kit, the outer boxes from the storage locations by comparing the assay kit collection code on each outer box in order to verify they are associated with the particular single assay kit. The kit collection code is readable by a human or a machine or both.

Another aspect of the disclosure provides a method of manufacturing an assay kit comprising inputting customer order information for a particular single assay kit into a database accessible by computer and retrieving from the database preloaded identification of reagents for the particular single assay kit; optionally displaying a location map for the particular assay kit indicating where, in an insert, a plurality of assay kit reagent containers comprising the reagents for the particular single assay are to be located, the insert comprising an upper surface, a bottom surface, and a plurality of through holes extending from the upper surface of the insert to the bottom surface of the insert, each of the through holes configured to hold a respective assay kit reagent container; preparing a loaded insert by providing the insert and providing the plurality of identified assay kit reagent containers and optionally disposing in accordance with the location map each of the assay kit reagent containers into a respective through hole of the plurality of through holes, each of the assay kit reagent containers or a subset thereof having a container bottom comprising a product code identifying the reagent within a particular container, the product code readable by a human or a machine or both through the respective through hole at the bottom surface of the insert; and inputting the product code on each container bottom within the loaded insert into the database and comparing the product code on each container with the location map to verify that the assay kit reagent containers in the loaded insert are loaded in accordance with the location map for the particular single assay kit. In an alternative practice, the location of where the containers should be loaded in the insert is not provided, e.g. the locations in the insert are not preloaded into the database, and the insert is merely loaded with the plurality of identified assay kit reagent containers, whereafter the product code is read and the locations of the assay kit reagent containers as loaded into the insert are inputted to the database for future access.

In another aspect, the disclosure is directed to a package for an assay kit comprising: a) an outer box; and b) one or more removable inner boxes, i) wherein the outer box comprises a window and at least one of the inner boxes comprises assay product information, and wherein the assay product information is viewable through the window; or ii) wherein the at least one of the inner boxes comprises an insert that comprises an upper surface, a bottom surface, and a plurality of through holes extending through the insert from the upper surface to the bottom surface, and wherein each of the plurality of through holes is shaped to individually hold a respective assay kit reagent container such that a product code located on all or a subset of the bottoms of the assay kit reagent containers is readable through the respective through hole at the bottom surface of the insert, and wherein the insert for at least one of the inner boxes is removable from the inner box, or comprises a portion that is removable from the inner box, and wherein the removable insert or the removable portion is adapted to be placed on a scanner to read the product code; or iii) both (i) and (ii). Inner box as used herein includes, without limitation, a box having an open top or upper side, e.g. and without limitation, a box with four vertical walls, a closed bottom, and an open top or upper side, e.g. a tray; and includes in this regard such boxes such as trays, and including trays of different size relative to each other, e.g. a smaller tray (or minor tray) as compared to a larger tray (a major tray).

### BRIEF DESCRIPTION OF THE DRAWINGS

FIG. 1 is a top view of an embodiment of an insert of the disclosure.

FIG. 2 is a cross sectional view of the insert FIG. 1 loaded with assay kit reagent containers.

FIG. 3 is a partial bottom view of the loaded insert of FIG. 2.

FIG. 4 is a perspective view of an embodiment of an inner box, e.g. tray, of the disclosure.

FIG. 5 is a perspective view of another embodiment of an inner box, e.g. tray, of the disclosure.

FIG. 6 is a perspective view of an alternative embodiment of an inner box, e.g. tray, of the disclosure having internal segments.

FIG. 7A is a top view of an embodiment of an inner box, e.g. tray, with two inserts, one of which is loaded with assay reagents; FIG. 7B is a top view of the two inserts showing through holes.

FIG. 8 is a perspective view of differently sized outer boxes for the package of the disclosure.

FIG. 9 shows differently sized inner boxes having storage temperature information thereon (only shown on inner boxes E and G in the figure).

FIG. 10 shows side and top views of an inner box, e.g. tray, with inserts in an embodiment of the disclosure.

FIG. 11 shows top views of three inner boxes, e.g. trays L, L1, L2 (which can each stand alone as separate embodiments; or can include any combinations of any of L, L1, L2 as distinct separate embodiments), with inserts in embodiments of the disclosure.

FIG. 12 shows side and top views of an inner box, e.g. tray, with inserts in an embodiment of the disclosure.

FIG. 13 shows top views of three inner boxes, e.g. trays N, L1 and L2, with inserts in embodiments of the disclosure.

FIG. 14 shows side and top views of an inner box, e.g. tray, with inserts in an embodiment of the disclosure.

FIG. 15 shows top views of three inner boxes, e.g. trays Q, L1, and L2, with inserts in an embodiment of the disclosure.

FIG. 16 is a perspective view of differently sized outer boxes for the package of the disclosure.

FIG. 17 shows top views of an inner box, e.g. tray, with inserts in an embodiment of the disclosure.

FIG. 18 shows top views of an inner box, e.g. tray, with inserts in an embodiment of the disclosure.

FIG. 19 shows top views of three inner boxes, e.g. trays T, L2, and L3, with inserts in an embodiment of the disclosure.

### DETAILED DESCRIPTION

The ensuing description is exemplary only and is not limiting to the scope of the disclosure. U.S. Pat. No. 8,770,471 is incorporated herein by reference in its entirety for all purposes.

Unless otherwise defined herein, scientific and technical terms used in connection with the present disclosure shall have meanings commonly understood to those of ordinary skill in the art. Unless otherwise required by context, singular terms shall include pluralities and plural terms shall include the singular. The articles “a” and “an” are used herein to refer to one or to more than one, i.e. to at least one. For example, “an element” means one element or more than one element.

The package, insert, inner box, and methods of the disclosure include a variety of designs and configurations. As used herein, the “product code” and the “assay kit collection code” are each a code that is machine readable, human readable, or both. In one practice it is an alphanumeric code. When machine readable, the product code and assay kit collection code can each individually comprise, e.g. a barcode or other scannable configuration. In one embodiment, the product code and the assay kit collection code each individually comprise a 2D or 3D data-matrix barcode or QRS code. The product code identifies the reagent, e.g. by the assay name or by the category of assay type, in a pertaining assay kit reagent container, and optionally which assay kit products employ such reagents and optionally location map layout rules for preparing a loaded insert as described herein. The assay kit collection code is uniquely associated with a particular single assay kit, e.g. the assay kit collection code is the same for outer boxes sold as including reagents and components, such as assay plates, for that particular single assay and facilitates identification and matching of reagents and components for a particular single assay kit when storing and re-assembling for use, among other things.

For example, in one practice, the product code is a bar code, e.g. a 2D or 3D data-matrix barcode, or QRS code, located on the bottom of assay kit reagent containers. As used herein, being located on the container bottom includes locations proximate the container bottom. The product code for a container can comprise, without limitation, information identifying the reagent residing within the container, and optionally information which can further comprise one or more or following: a description of that particular assay kit, including e.g. its use and an analyte list for that for that particular assay kit, the manufacturing lot number for that reagent, a catalog number for the particular assay kit to which that reagent pertains, a numbered spot map representation, and a legend that links to a spot map via a spot number wherein the legend contains, without limitation, information on the reagent for a particular spot, or the legend contains a blank if the spot is not used in that particular assay or was not purchased or otherwise provided by a supplier to the customer of the assay. Assay kit reagent containers include without limitation those known in the art, e.g. vials, tubes, bottles, jars, small boxes, and other vessels suitable for holding and/or transporting assay kit reagents, and may comprise different shapes and/or sizes. Small boxes may contain other assay kit reagent containers, e.g., vials, tubes, and bottles. The assay kit reagents within the containers may be the same or different; assay kit reagents, as used herein, include without limitation those known in the art, e.g. antibodies, controls, diluents, buffers, calibrators, and other chemical or biological materials and the like. The assay kit reagent containers may be organized such that those needing storage at the same temperature, e.g. room temperature (RT), low temperature (e.g. 4 degrees C.), or frozen (e.g., −10 or −70 degrees C.), are packaged together or are adapted to be packaged together. Assay kit components, as used herein, as used herein include non-reagent components such as, without limitation those known in the art, including e.g. assay plates, cartridges, and the like.

In a non-limiting embodiment, a package for a particular assay kit is provided, the package comprises a outer box, which includes containers of any shape, e.g. without limitation cuboid shapes such as rectangles and squares, that has an outer sidewall on which is located assay product information, e.g. without limitation, brand name and/or category of type of the particular assay kit contained therein, logos, the assay kit collection code, etc. In one practice, the package further comprises an outer box that has a sidewall with an optional window therethrough. The outer box is adapted to or disposed within the outer box to display the assay product information through the window.

An insert is disposed within the outer box, the insert being configured to hold a plurality of assay kit reagent containers. The insert can be comprised of any material suitable to hold the assay kit reagent containers in a manner consistent with movement incurred during transport and shipping. For example, the insert can comprise a polymer, such as a foam, including open-cell foam e.g. comprising soft high density polyethylene foam, and closed-cell foam, e.g. solid foam comprising polyurethane; inserts comprised of other suitable materials such as without limitation cardboard and the like are also contemplated. The insert in one practice comprises an upper surface that optionally can be substantially planar, a bottom surface that optionally can be substantially planar and optionally substantially parallel with the upper planar surface, and a plurality of through holes that extend completely through the insert from the upper surface to the bottom surface. Each of the plurality of through holes is shaped to individually hold a respective assay kit reagent container.

In one practice, the through holes are sized to snugly hold a respective assay kit reagent container; in another practice, the insert comprises a transparent bottom on which the assay kit reagent containers sit, in which practice the through holes may be adapted to a snug or non-snug fit with a respective assay kit reagent container. Each assay kit reagent container or a subset of assay kit reagent containers has a container bottom on which a product code is located. The insert is shaped and/or designed such that the product code located on each container bottom or subset of same having the product code is readable through the respective through hole at the bottom surface of the insert. The product code can be printed directly on the container bottom or be on a label or other identifier or tag affixed to the container bottom. In one practice, the insert is adapted to be placed on a scanner, e.g. a flat bed scanner, to read the product codes on the pertaining container bottoms.

In another embodiment, the insert is disposed within an inner box, e.g. tray, sized to fit within and be removable from an outer box. In one practice, the removable inner box, e.g. tray, comprises a closed bottom. In another practice, the removable inner box, e.g. tray, comprises an open bottom area, the tray comprising an inwardly-extending peripheral lip on which an outer edge of the bottom surface of the insert sits, the inwardly extending peripheral lip defining an open bottom area, through which open bottom area the product code located on each of the plurality of container bottoms is readable by human, machine or both through the respective through hole at the bottom surface of the insert. In another practice of this embodiment, the removable inner box, e.g. tray, comprises a transparent bottom on which the insert sits, e.g. the bottom is comprised of a transparent material such as a clear plastic through which the product code on the bottoms of the assay kit reagent containers is readable through the respective through hole at the bottom surface of the insert by human, machine or both. Such transparent materials include without limitation, cellophane and the like. In another separate practice, the insert itself can optionally have a transparent bottom, including without limitation, one comprised of the transparent materials aforesaid. In one particular embodiment, the removable inner box, e.g. tray, comprises a base which has upwardly-extending sidewalls that define an interior volume within which the insert is disposed, the base may be closed, or open. If open, the base can comprise a transparent material through which the product code located on each of the plurality of container bottoms is readable through the respective through hole at the bottom surface of the insert; or it can comprise an open bottom, e.g., the base comprises an inwardly directed lip extending around the periphery of the base, which inwardly directed lip defines the open bottom area through which the product code located on each of the plurality of container bottoms is readable through the respective through hole at the bottom surface of the insert. The insert may optionally have a peripheral sidewall that is in substantially continuous contact with the upwardly extending inner box, e.g. tray, sidewalls. In a particular embodiment, the inner box, e.g. tray, comprises cardboard with a full cardboard bottom; the inner box, e.g. tray, in this regard can be a unitary structure formed from a single piece of cardboard, or be comprised of different cardboard elements attached to form the inner box, e.g. tray. The package can comprise an outer box optionally having a window through which the assay product information located on the inner box, e.g. tray, e.g. printed on a tray outer sidewall, is viewable.

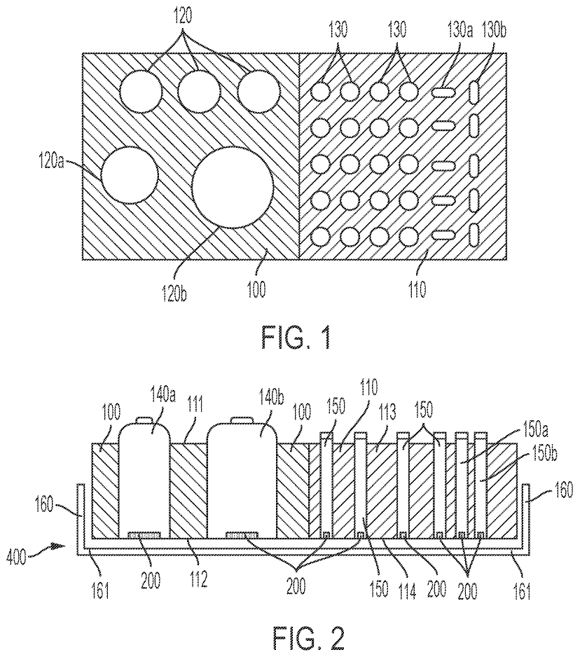

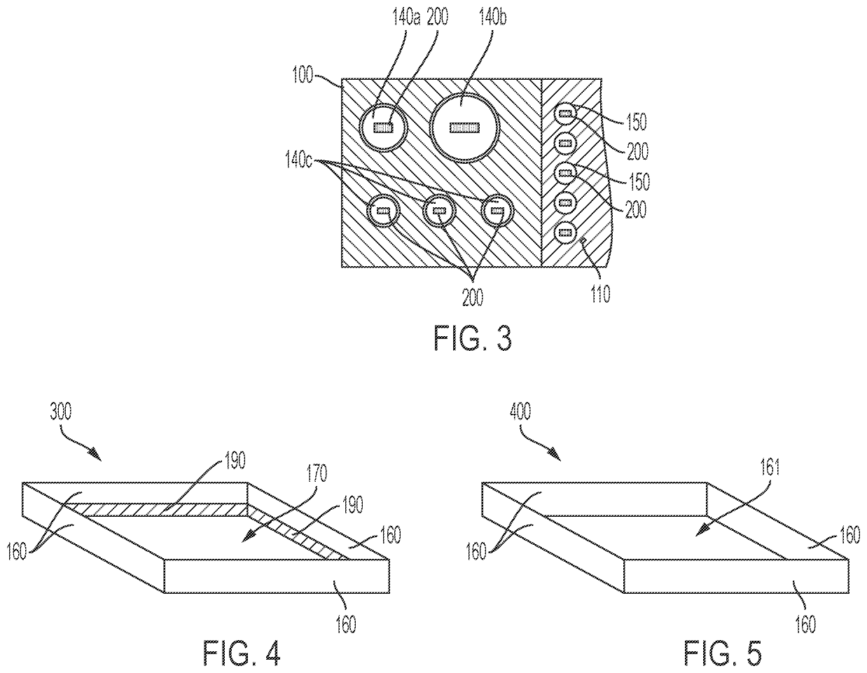

FIGS. 1, 2, 3, 4, and 5 show embodiments of an insert and an inner box, e.g. tray, contemplated by the disclosure. FIGS. 1, 2, 3, 4, and 5 are not to scale and are schematic and representative representations only and are not limiting to the disclosure. The figures depict two inserts 100 and 110 abutted together in a side by side configuration (the height of inserts 100 and 110 can vary one to the other and are depicted as approximately the same as representative only, e.g. insert 110 can be much thinner than 100). Insert 100 comprises an upper surface 111 and a bottom surface 112 each of which are substantially planar and substantially parallel to each other, and a plurality of through holes of different sizes 120, 120a, 120b which extend completely through the insert 100 from the upper surface 111 to bottom surface 112. Similarly insert 110 comprises an upper surface 113 and a bottom surface 114 each of which are substantially planar and substantially parallel to each other, and a plurality of through holes of different sizes and different shapes 130 130a, 130b which extend completely through the insert 110 from the upper surface 113 to bottom surface 114. As depicted in FIGS. 2 and 3, assay kit reagent containers 140a, 140b, and 140c are respectively disposed in through holes 120a, 120b, and 120 whereas assay kit reagent containers 150, 150a, and 150b are disposed in through holes 130, 130a and 130b respectively. As depicted in FIG. 3, each of the assay kit reagent containers has an product code 200 located on the container bottom and readable through the though hole of the bottom surface of the insert. In other embodiments, a subset of the assay reagent containers comprises a product code 200 specific to each assay reagent. FIGS. 2 and 3 further depict one or both of the inserts being removably disposed within a tray, e.g. tray 400 of FIG. 5, which has upwardly extending tray sidewalls 160 that define an interior volume within which insert 100 is disposed, as well as insert 110. Tray 400 comprises closed bottom 161 on which inserts 100, and 110 sit, as shown in FIG. 2. One or both of inserts 100 and 110 are removable from tray 400 and adapted to be scanned, e.g. to be placed on a flat bed scanner which can scan the product codes 200 as shown in FIG. 3. FIG. 4 depicts an alternative embodiment wherein tray 300 comprises upwardly extending tray sidewalls 160 that define an interior volume in which the inserts are disposed and which comprises inwardly directed peripheral lip 190 on which the outer edges of the inserts sit and which define an open area 170 through which the product codes are readable, e.g. the tray containing the insert can be placed on a flat bed and the product codes read. In another practice, the open area 170 can be covered with a transparent material on which the insert sits and through which the product codes 200 are readable (not shown). It will be understood that while two inserts 100 and 110 are shown, one or more than two inserts may be employed, including in a single or multiple trays and that each insert may be of different shape, size, and thickness. For example, insert 110 may include assay reagent components that have shorter height in cross section shown in FIG. 2 (FIG. 2 is not to scale and is representative only) than the assay reagent components in insert 100 and its height in cross section may thus be lower than that of insert 100. It will also be understood that while FIG. 2 shows fully loaded inserts 100 and 110, the one or more inserts may be less than fully loaded with assay reagent containers as needed for the type of assay or the number of assays (e.g., singleplex versus multiple or number of assay modules) included in the kit.

A method of manufacturing an assay kit, including the preparation of a fully or partially loaded insert for an assay kit, is provided where an order from a customer for a particular assay kit is received. In one embodiment, the method comprises inputting, e.g. by an operator, the customer order information pertaining to the particular assay kit into a database that is accessible by computer. In one practice, the database comprises information about the particular assay kit, including without limitation, the identification of which assay kit reagents and which assay kit components are associated with that particular assay kit, where the related assay kit reagent containers are located, what temperatures they are stored at, what lot numbers are assigned to them, and the number of assay kit reagent containers and components that are available for that particular assay kit. Assay kit components include, without limitation, non-reagent components, such as, e.g. assay plates, cartridges and the like. The method may further comprise a step of displaying a location map, e.g. on a graphic display, which indicates where in an insert the assay kit reagent containers for that particular assay kit are to be located. The insert, as herein described, comprises an upper surface, a bottom surface, and a plurality of through holes extending from the upper surface of the insert to the bottom surface of the insert, wherein the through holes are configured to individually hold a respective assay kit reagent container, the location map identifying into which hole or holes particular assay kit reagent containers are to be disposed. For example, the map may indicate for a plurality of assay kit reagent containers comprising antibodies that they are to be located together in adjacent through holes, and that other assay kit reagent containers for buffers are to be located together elsewhere in the same insert. The method comprises a step of preparing a loaded (fully or partially loaded) insert, in accordance with the location map, by means of providing the insert, which may be of uniform size for all assay kits or selectively sized for a particular assay kit, and providing the plurality of assay kit reagent containers associated with that particular assay kit, and disposing, manually or automatically, each of the assay kit reagent containers into a respective through hole identified by the location map as being for that reagent container. In an alternative practice, the location of where the containers should be located in the insert is not provided, e.g. the locations in the insert are not preloaded into the database, and the insert is merely loaded with the plurality of identified assay kit containers, whereafter the product code is read and the locations of the assay kit reagent containers as loaded into the insert are inputted to the database for future access.

Each of the assay kit reagent containers has a container bottom on which a product code is located, or a subset of the bottoms has a product code (e.g. a QRS code), the product code e.g. is printed directly on the container bottom, or is printed on a label that is affixed to the container bottom. The product code can either be extant on the container bottom before or when the reagent container is provided for assembly, or can be directly printed onto the container bottom or affixed by a label during the method of assembling. The assay kit reagent containers are disposed into the respective through holes of the insert such that the product code at the container bottom, for containers comprising the product code, is readable by a human or a machine or both through that respective through hole at the bottom surface of the insert. Container bottom as used herein indicates the bottom surface per se of the container, or an area sufficiently proximate to the actual bottom surface to permit reading of the product code, e.g. a puck applied to the bottom of a tube or the sidewall area immediately adjacent the bottom that is viewable from the underside of the insert.

The method further comprises inputting the product code on each container bottom comprising the product code within the loaded insert into the same database and/or different database and comparing the product code on the containers with the preloaded information in the database to verify that the assay kit reagent containers in the loaded insert are for the particular single assay kit. Inputting may be manual, e.g. by an operator logging in the product code on each container by hand, or by scanning to automatically input the product code on the containers, e.g. by a hand held scanner, or by a flat bed scanner or similar scanner wherein the loaded insert is placed upon the flat bed scanner which then scans the entirety of the loaded insert bottom surface and the product codes on the container bottoms readable via the bottom of the insert through holes. The scanned product codes are compared, e.g. matched by computer to the product code information in the database for that particular assay, to verify that the assay kit reagent containers in the loaded insert are for that particular single assay kit. Any container whose product code does not match those for the particular assay kit is identified e.g. by a warning on a graphic display or by other means identifying the causative container or containers in the loaded insert so that it may be removed and replaced by a container having the proper code for that particular assay kit. As understood throughout this disclosure, information that is scanned can be stored locally or via a network or via cloud.

In another aspect, the method further comprises retrieving from the same or different database identification of assay kit components, such as assay modules (e.g. assay plates or cartridges), or product literature, associated with the particular single assay kit being assembled, and providing these components for inclusion into the assembly process. In one practice, each assay kit component has thereon a component code that is readable by a human or a machine or both and identifies what the component is and to which assay kit product it is associated with. This aspect of the method comprises inputting the component code on each assay kit component into the database and comparing the component code on each assay kit component with the information in the database to verify that each assay kit component is for the particular single assay kit. Any component whose component code does not match that for the particular assay kit is identified e.g. by a warning on a graphic display or by other means identifying the causative component or components so that they may be replaced by components having the proper code for that particular assay kit. The particular single assay kit is then assembled by combining the verified kit components and the verified loaded insert into one or more packages, including a package as disclosed herein, for shipping to the customer identified on the customer order information.

The disclosure further provides a method for storing and retrieving an assay kit comprising the steps of providing to a customer outer boxes as defined for a particular single assay kit. The outer boxes may be shipped to the customer together or separately, including over separate periods of time. Each outer box comprises an outer sidewall on which is located by printing or label or other means, an assay kit collection code that is human readable, machine readable or both and is uniquely associated with the particular assay kit that has been shipped. The outer box contains an insert, including as herein described, disposed within it, the insert holding a plurality of assay kit reagent containers comprising the same or different reagents for that assay kit. In one aspect of the method, information from the assay kit collection code located on each outer box is inputted by hand or by scanning into a database accessible by a computer, e.g. to confirm receipt, create an inventory etc.

In one aspect, the method entails storing respective received outer boxes at storage locations, which storage locations can be the same location or different locations, including locations having different storage environments depending on the reagents contained within the assay kit reagent containers or inner boxes. These different environments can be in the same location or at multiple, physically separated locations, e.g. spaces at different temperatures such as multiple different freezers or refrigerators or spaces at RT. In one practice, the method comprises inputting information about the storage locations into the database and associating the information about the storage locations with the assay kit collection code or on one more of the product codes. For example, the storage locations can comprise labels, such as located on or near freezer doors, shelves etc. identifying the location, e.g. by address or name or other nominator, and/or the portion of the location, including optionally the environmental condition of that particular storage location, where the respective outer box is stored. The customer can hand input or scan the labels at the storage location.

The method further comprises retrieving, when ready to assemble, e.g. use, the particular assay kit, the respective outer boxes from their individual storage locations by comparing the assay kit collection code on each outer box, by scanning wherein the comparison is done by computer, or visually by the customer, in order to verify that all outer boxes retrieved are for that particular assay kit. In one aspect, if the storage location information has been inputted into the database as aforesaid, the customer can access the database, identify the storage locations of the respective outer boxes, optionally including the environmental conditions of storage, to retrieve the outer boxes and retrieve them in a determined sequence, e.g. those outer boxes stored at low temperature may be retrieved last. In one practice, when the outer boxes are retrieved from their respective locations, the retrieval is inputted into the database by hand or by scanning to denote that a particular outer box has been removed, optionally with the time and date, and the inventory database adjusted to reflect this fact accordingly. The assay kit can then be assembled with the reagents contained within the assay kit reagent containers that have been provided in the outer boxes. In another practice, the assay kit collection code is also located on the insert, which insert can be removed from the outer box, the insert then being stored and retrieved as herein described. In one practice, the information from the assay kit collection code is used to create an inventory in the database of the outer boxes and the locations of same for that particular assay kit. The assay kit can then be assembled, e.g. for use, from the retrieved reagents after the assay kit collection code has been verified for that particular assay kit.

In another aspect, the disclosure provides a package for an assay kit comprising: a) an outer box; and b) one or more removable trays, i) wherein the outer box comprises a window and at least one of the trays comprises assay product information which can comprise assay name or category of assay type, and wherein the assay product information is viewable through the window; or ii) wherein the at least one of the trays comprises an insert, which can comprise foam, that comprises an upper surface, a bottom surface, and a plurality of through holes extending through the insert from the upper surface to the bottom surface, and wherein each of the plurality of through holes is shaped to individually hold a respective assay kit reagent container such that a product code located on all or a subset of the bottoms of the assay kit reagent containers is readable through the respective through hole at the bottom surface of the insert, and wherein the insert for at least one of the trays is removable from the tray, or comprises a portion that is removable from the tray, and wherein the removable insert or the removable portion is adapted to be placed on a scanner to read the product code; or iii) both (i) and (ii).

In one aspect of this practice, at least one insert is not removable; and the through holes for the at least one insert that is not removable are shaped to hold one or more bottles of the same or different shape, e.g. bottles of 25 ml volume or greater; in one embodiment in this regard, the bottle bottoms do not comprise the product code. In another aspect, the through holes for the removable insert or the removable portion are shaped to hold a plurality of vials or tubes or both, the vials and tubes of the same or different shape; and in one practice, the bottoms of the plurality vials or tubes each individually comprise the product code. The outer box and/or at least one of the trays and/or at least one of inserts can comprise storage temperature information.

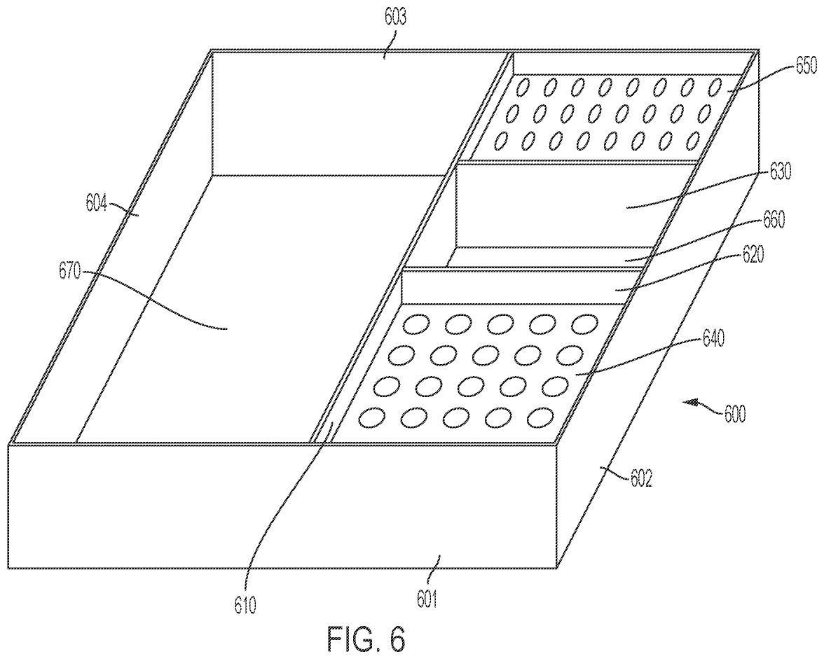

In another practice, the outer box comprises one or more internal walls configured to create one or more internal segments within the outer box; the one or more internal walls are integral to the outer box or are configured to be removable from the outer box. In this practice, at least one tray with insert is disposed within a respective internal segment; that at least one insert with tray can be removable from the respective internal segment. More than one removable insert can be disposed within the respective segment, and in one instance, the more than one removable inserts with tray are disposed side by side within the respective segment, or the more than one removable inserts with tray are stacked on top of one another within the respective segment. Optionally, one or more assay modules, e.g. assay plates or cartridges, are disposed within a respective internal segment. Likewise, one or more inner boxes with or without inserts are located within a respective internal segment. In one particular embodiment, one or more bottles are disposed within a first respective internal segment or within an insert that is not removable, the insert that is not removable disposed within the first respective internal segment; and wherein one or more vials, tubes, or both are disposed within the at least removable insert or removable portion, the at least removable insert or removable portion is disposed within a second internal segment; and one or more assay plates disposed within a third internal segment. As shown in the representative practice FIG. 6, tray 600 comprises, in addition to its external walls 601, 602, 603, 604, internal walls 610, 620, and 630 which are configured to create four internal segments: 670 and 660; the remaining two internal segments shown as having inserts 640 and 650 respectively disposed therein.

In the embodiments described herein, the inner boxes, e.g. trays, can be of different sizes and configured to be stackable. Likewise, the inserts can be of different sizes and configured to be stackable. The through holes can be of different or varying sizes for a given insert or within the same insert. The outer boxes can come in various sizes and the package or system of packaging is optimized so that the fewest number of outer boxes, inner boxes, and inserts are required for a given assay product, plurality of sizes of assay product, or plurality of or totality of assay types within a manufacturers' product offering.

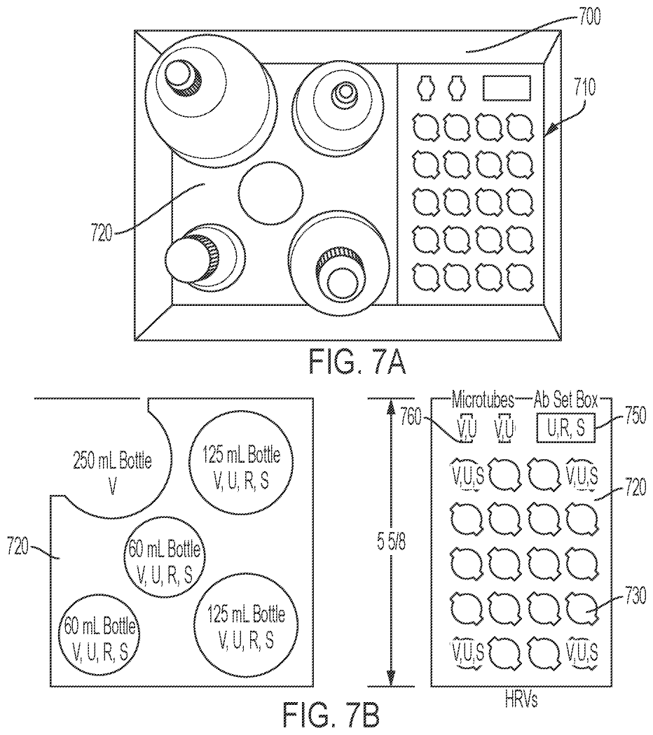

FIG. 7A shows tray 700 with two inserts 710, and 720, with 720 having five bottles of differing sizes disposed in same. FIG. 7B shows the insert 720 and through holes of different sizes and insert 710 with through holes with shapes 730, 760 and 750. Insert 710 is removable and adapted to be scanned.

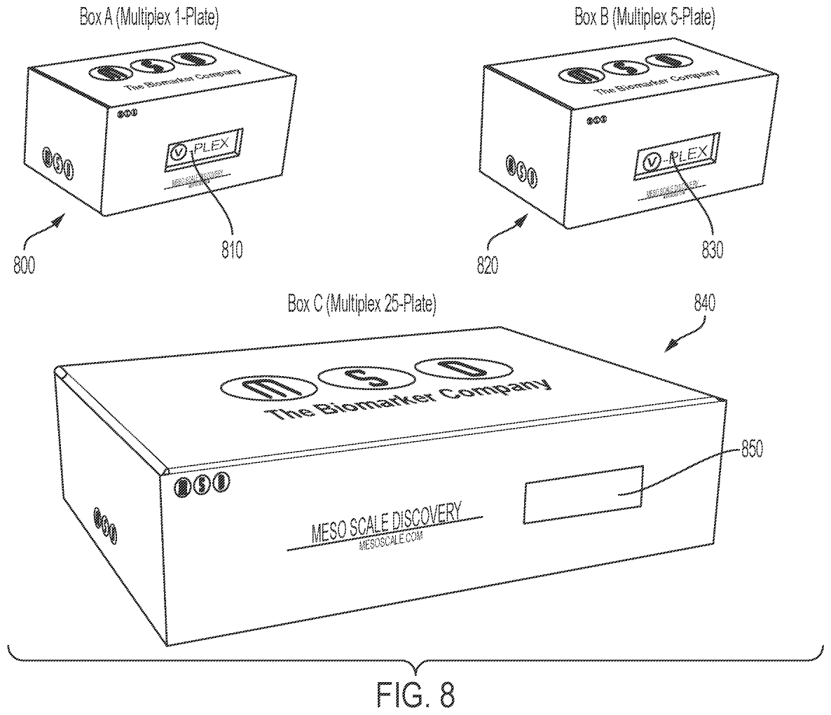

FIG. 8 shows embodiments of outer boxes of different sizes 800, 820, 840, each having a respective window 810, 830, 850 (quantities as indicated).

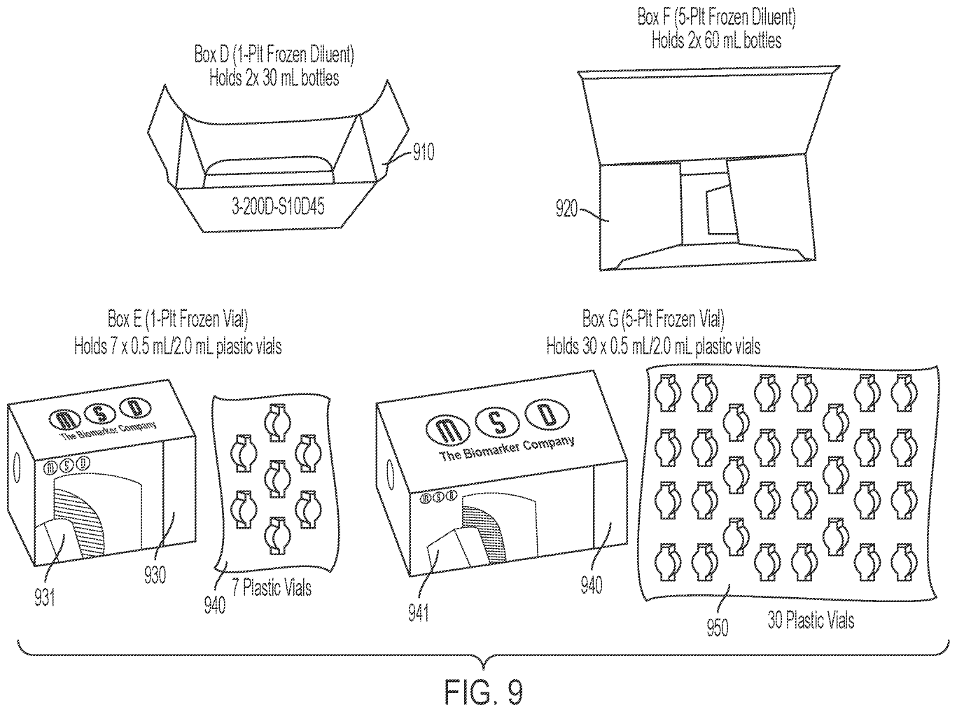

FIG. 9 shows an embodiment of differently sized inner boxes 910, 920, 931, and 941 suitable for frozen assay reagents, e.g. diluents that need storage at −10° C. or −70° C. and every temperature in between; in which inner boxes 931 and 941 have storage temperature information 930 and 940, e.g. color coded and text showing needed storage at −70° C., and inserts 940 and 950 holding plastic vials whose contents require such frozen storage. Types and quantities of containers as indicated. Although not shown, inner boxes 910 and 920 may also have storage temperature information, e.g. color coded and text showing needed storage at −10° C.

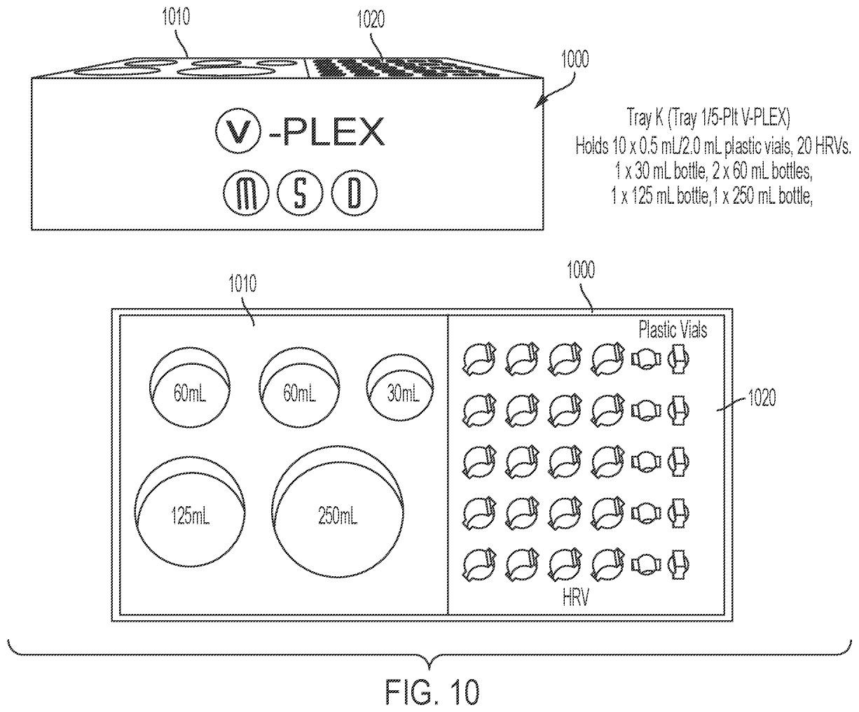

FIG. 10 shows an embodiment of a tray 1000 with two inserts, one insert 1010 for differently sized bottles, and a side by side insert 1020 for plastic vials and high recovery vials (HRV). Types and quantities of containers as indicated.

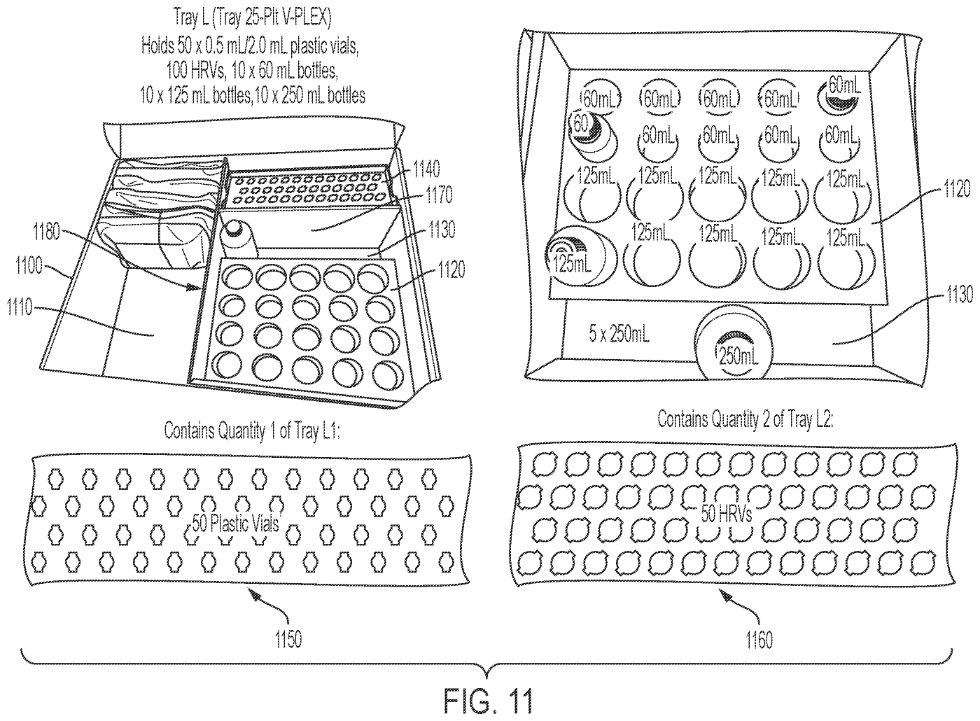

FIG. 11 shows an embodiment of a package comprising outer box 1100 with inner wall 1180. Insert 1120 is disposed within tray 1130 (tray L), but is of smaller size than tray 1130, the space unoccupied by insert 1120 is available for packing of additional containers, such as one or more bottles as shown, or other assay kit components. Segment 1110 contains no insert and is available for packing other assay kit components, such as assay modules (e.g. plates or cartridges) or inner boxes other than trays. Segment 1140 is formed when tray L is included in the outer box 1100 and comprises multiple inserts with trays stacked on top of one another, in the case of FIG. 11, one insert 1150 for plastic vials, and two inserts 1160 for HRVs. Types and quantities of containers as indicated.

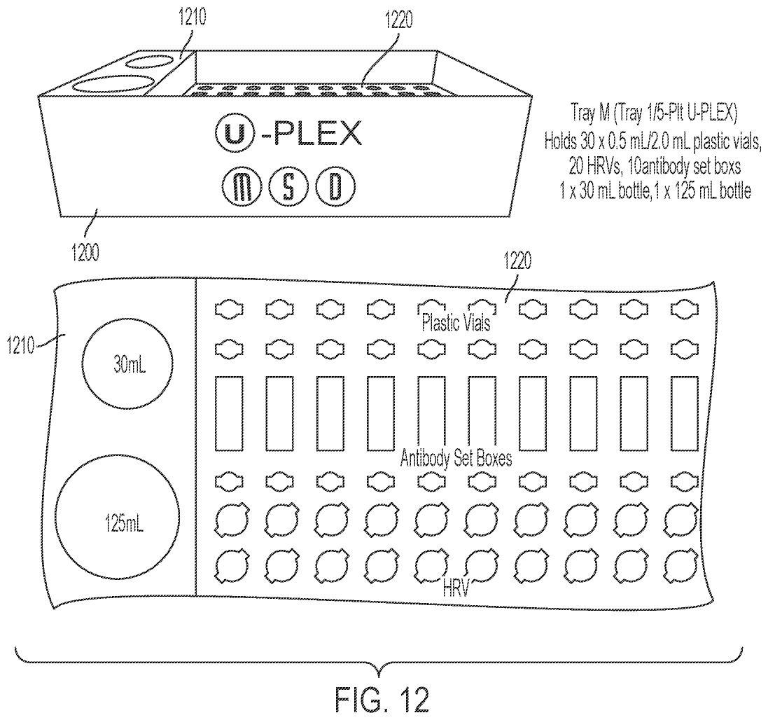

FIG. 12 shows an embodiment of a tray 1200 with two inserts, one insert 1210 for differently sized bottles and insert 1220 for plastic vials, antibody set boxes, and HRVs. In this embodiment, insert 1220 does not have the same vertical dimension as insert 1210, insert 1220 being thinner and not reaching the top of tray 1200. Types and quantities of containers as indicated.

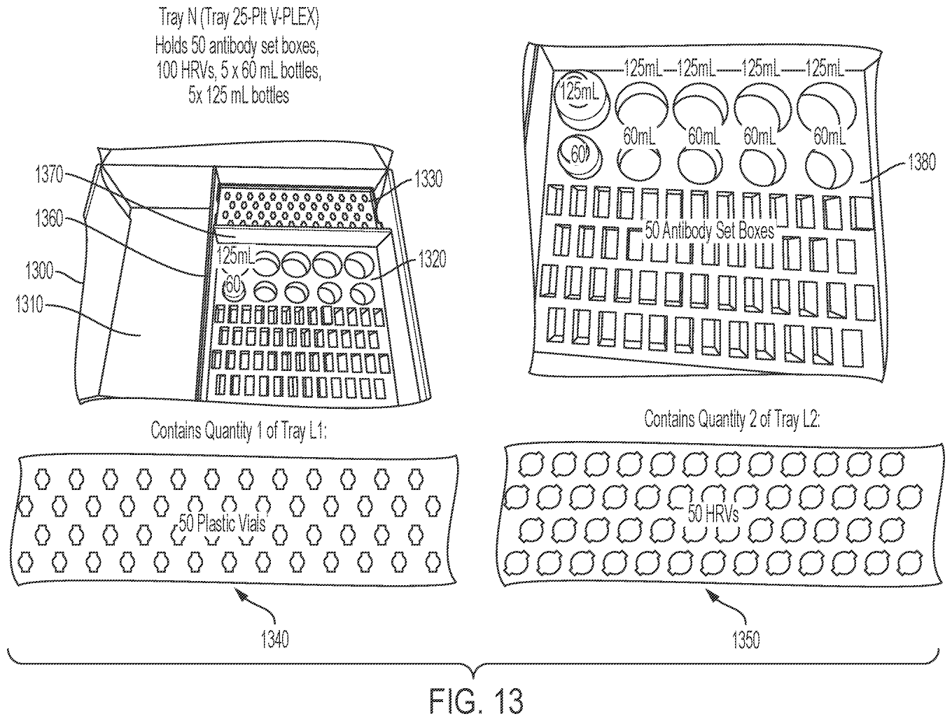

FIG. 13 shows an embodiment of a package comprising an outer box 1300 with inner wall 1360. Insert 1380 is fully disposed within tray 1320 (tray N) and is of unitary construction configured to contain bottles of various sizes, and antibody set boxes. Segment 1310 contains no insert and is available for packing other assay kit components, such as assay modules (e.g. plates or cartridges) or inner boxes or other trays. Segment 1330 is formed when tray N is included in outer box 1300 and comprises multiple inserts with trays stacked on top of one another, in the case of FIG. 13, one insert 1340 for plastic vials, and two inserts 1350 for HRVs. Types and quantities of containers as indicated.

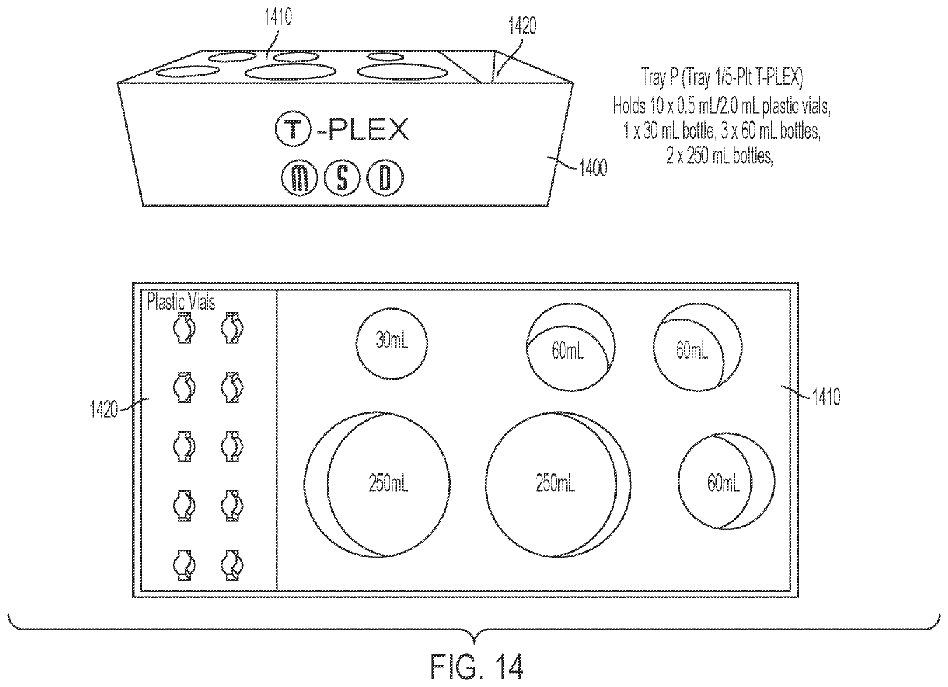

FIG. 14 shows an embodiment of a tray 1400 with two inserts, one insert 1410 for differently sized bottles and insert 1420 for plastic vials. In this embodiment, insert 1420 does not have the same vertical dimension as insert 1410, insert 1420 being thinner and not reaching the top of the tray 1400. Types and quantities of containers as indicated.

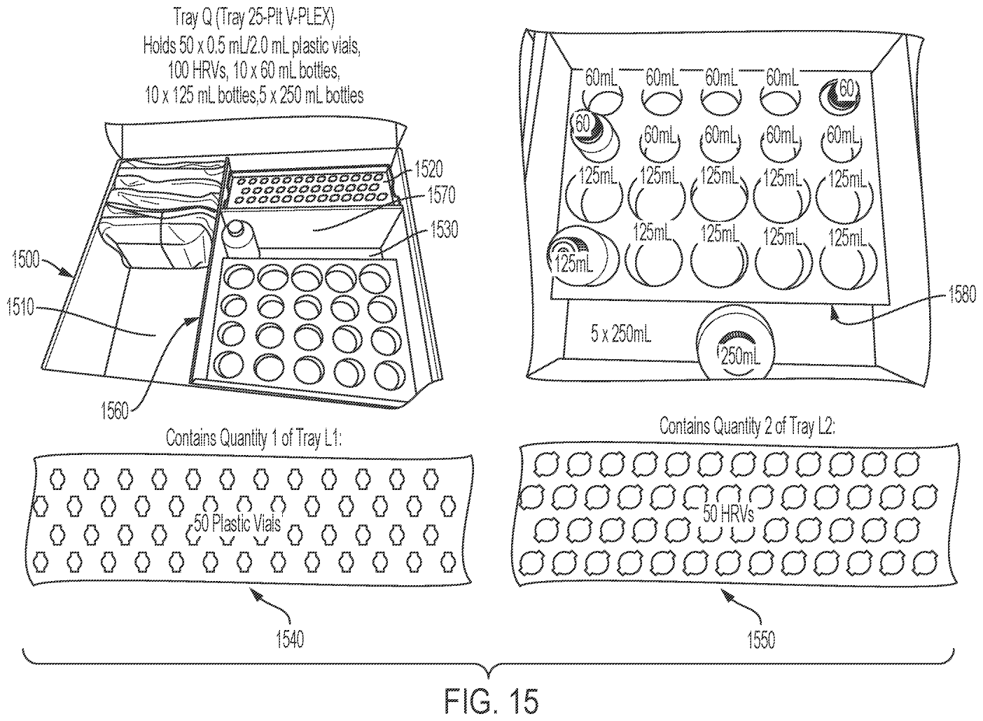

FIG. 15 shows an embodiment of a package comprising outer box 1500 with internal wall 1560. Insert 1580 is disposed within tray 1530 (tray Q), but is of smaller size than tray 1530, the space unoccupied by insert 1580 is available for packing of additional containers, such as one or more bottles as shown, or other assay kit components. Segment 1510 contains no insert and is available for packing other assay kit components, such as assay modules (e.g. plates or cartridges). Segment 1520 is formed when tray Q is included in outer box 1500 and comprises multiple inserts with trays stacked on top of one another, in the case of FIG. 15, one insert 1540 for plastic vials, and two inserts 1550 for HRVs. Types and quantities of containers as indicated.

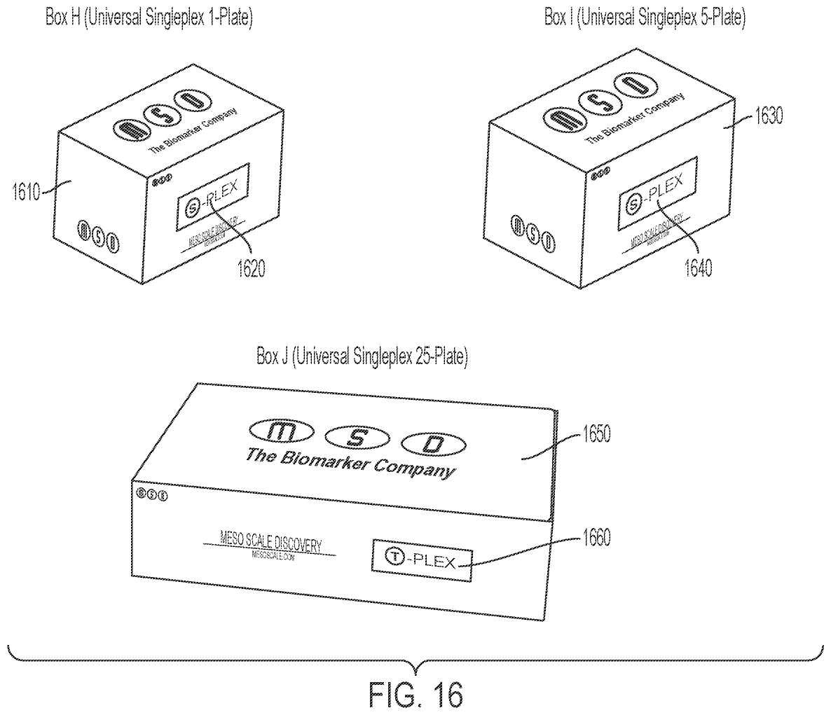

FIG. 16 shows outer boxes 1610, 1630, and 1650 of different sizes each having a respective window 1620, 1640, 1660.

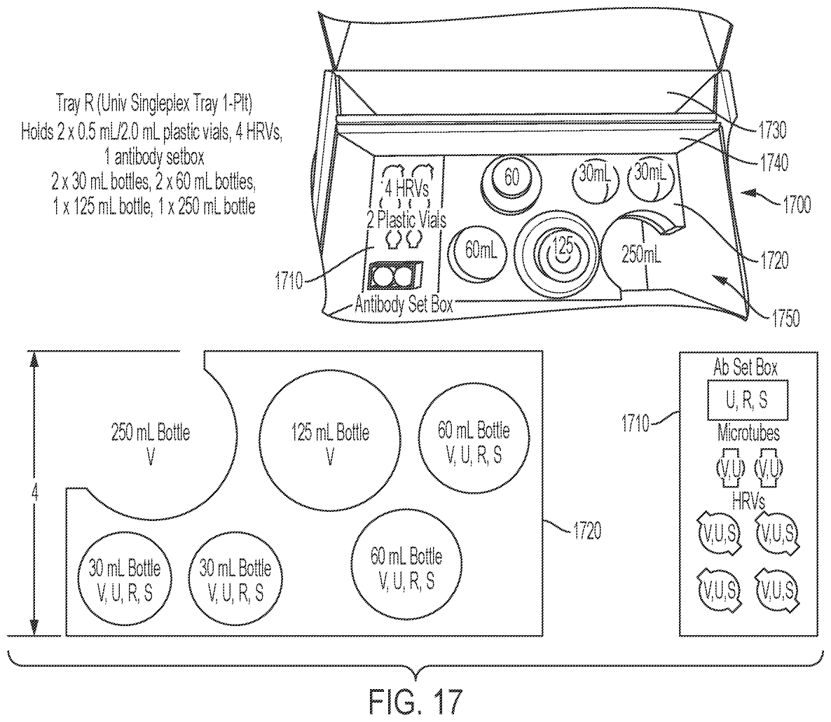

FIG. 17 shows an embodiment of package comprising an outer box 1700 with internal segments 1730 and 1750 being formed by internal wall 1740 (a double wall construction for 1740 being shown). Segment 1730 is unoccupied by an insert and is available for packing of other assay kit components such as assay modules (e.g. assay plates or cartridges). Inserts 1710 and 1720 are disposed side by side. Insert 1720 is for differently sized bottles and insert 1710 is for plastic vials (e.g., microtubes), antibody set boxes and HRVs. Types and quantities of containers as indicated.

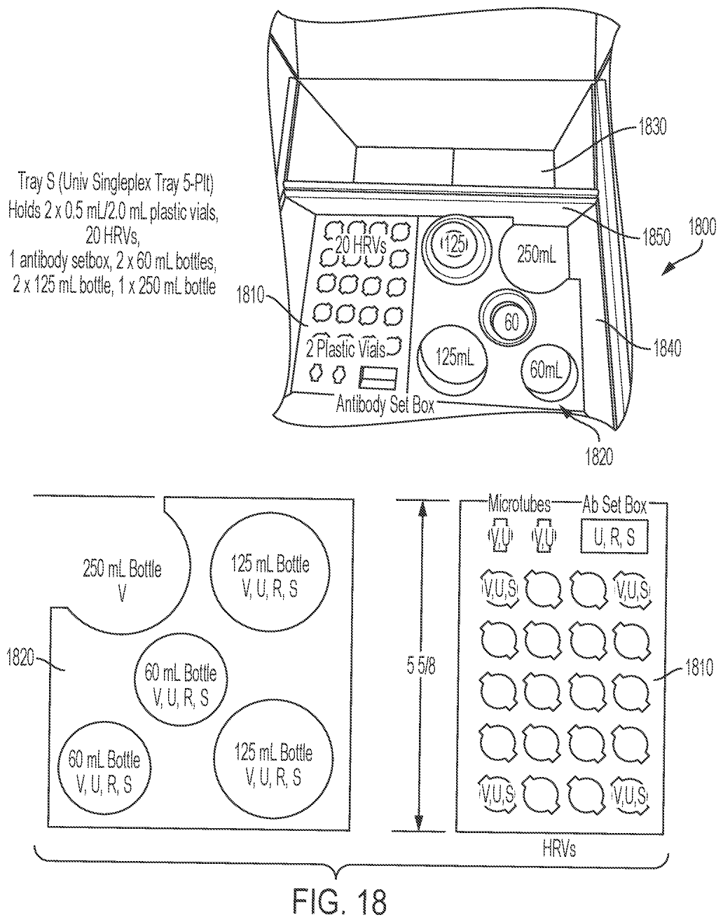

FIG. 18 shows an embodiment of package comprising an outer box 1800 with internal segments 1830 and 1840 being formed by internal wall 1850 (a double wall construction due to tray 1840 and internal wall 1850 being shown next to each other). Segment 1830 is unoccupied by an insert and is available for packing of other assay kit components such as assay modules (e.g., assay plates or cartridges). Inserts 1820 and 1810 are disposed side by side. Insert 1820 is for differently sized bottles and insert 1810 is for plastic vials (e.g. microtubes), an antibody set box and HRVs. Types and quantities of containers as indicated.

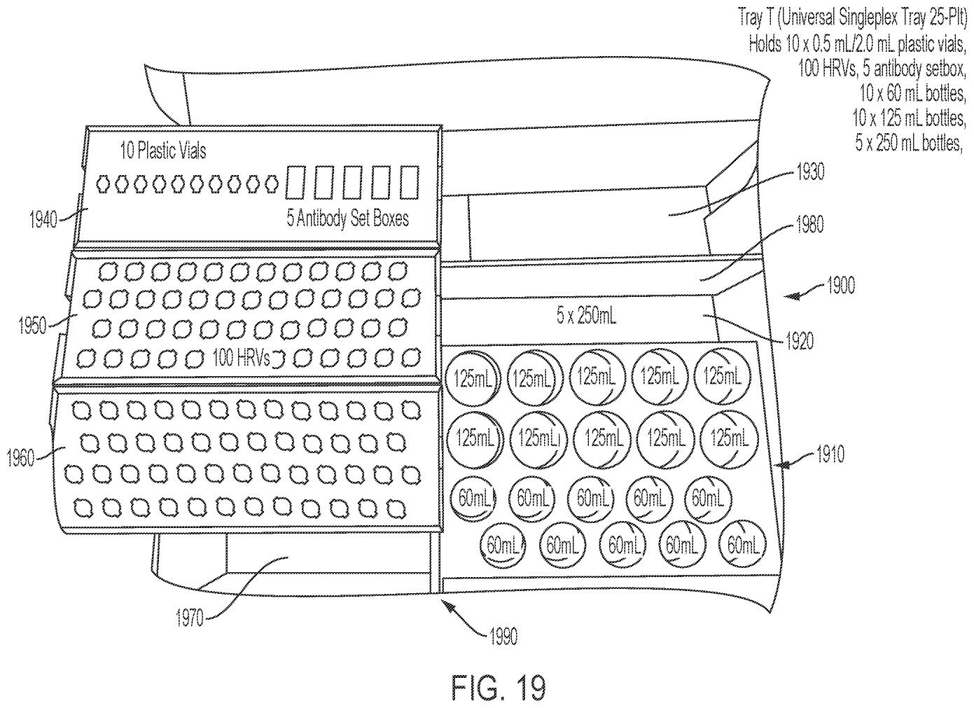

FIG. 19 shows an embodiment of a package comprising outer box 1900 with internal wall 1990. Insert 1910 is disposed within tray 1920 (tray T), but is of smaller size that tray 1920, the space unoccupied by insert 1910 is available for packing of additional containers, such as one or more bottles. Segment 1970 is unoccupied by an insert and is available for packing of other assay kit components, such as assay modules (e.g. plates or cartridges). Segment 1930 is formed when tray T is included in the outer box 1900 and comprises multiple inserts with trays stacked on top of one another, in the case of FIG. 19 insert 1940 for plastic vials and antibody set boxes, and two inserts 1950 and 1960 for HRVs. Types and quantities of containers as indicated.

Additional embodiments of the package are described herein are provided in the attached appendix the contents of which are part of this application.

1. A package for a particular assay kit comprising:
a tray having assay product information on an outer sidewall;
an insert disposed within the tray, the insert configured or adapted to hold a plurality of assay kit reagent containers comprising the same or different reagents, wherein each assay kit reagent container has a container bottom, wherein all or a subset of each assay kit reagent container bottom comprises a product code that is readable by a human, a machine, or both and identifies the reagent within that particular container, wherein the insert comprises an upper surface, a bottom surface, and a plurality of through holes extending through the insert from the upper surface to the bottom surface, and wherein each of the plurality of through holes is shaped to individually hold a respective assay kit reagent container such that the product code located on each container bottom is readable through the respective through hole at the bottom surface of the insert.

2.-10. (canceled)

11. A loaded insert comprising:
an insert comprising an upper surface, a bottom surface, and a plurality of through holes extending from the upper surface of the member to the bottom surface of the insert; and
a plurality of assay kit reagent containers comprising the same or different reagents, each of the plurality of assay kit reagent containers disposed within a respective through hole of the plurality of through holes, wherein all or a subset of the plurality of assay kit reagent containers has a container bottom on which is located a product code identifying the reagent within that particular container, and wherein the product code is readable by a human or a machine or both through the respective through hole at the bottom surface of the insert.

12.-15. (canceled)

16. A method of preparing a loaded insert for an assay kit comprising:
providing a plurality of assay kit reagent containers associated with a particular assay kit, the plurality of assay kit reagent containers comprising the same or different reagents, wherein each or a subset of the plurality of assay kit reagent containers have a container bottom on which is located a product code identifying the reagent within that particular container, the product code readable by a human or a machine or both;
providing an insert that comprises an upper surface, a bottom surface, and one or more through holes extending from the upper surface of the insert to the bottom surface of the insert;
preparing a loaded insert by disposing each of the plurality of assay kit reagent containers into a respective through hole of the plurality of through holes such that the product code on each container bottom or subset thereof comprising the product code is readable through the respective through hole at the bottom surface of the insert;
inputting the product code on each container bottom comprising the product code into a database accessible by a computer, the database comprising information about reagents for the particular assay kit; and
comparing the product code on each container comprising the product code with the information in the database about the reagents for the particular assay kit to verify that the reagents contained within the plurality of assay kit reagent containers are for the particular single assay kit.

17.-20. (canceled)

21. A method for storing and assembling an assay kit comprising
(i) providing, to a customer, outer boxes for a particular assay kit, wherein each outer box comprises (a) an outer sidewall displaying an assay kit collection code that is human readable, machine readable or both and is uniquely associated with that particular assay kit, and (b) an insert disposed within each outer box, the insert holding a plurality of assay kit reagent containers comprising the same or different reagents;
(ii) storing respective outer boxes at storage locations which can be the same or different depending on the reagents contained within the assay kit reagent containers for a respective product box; and
(iii) retrieving, when ready to assemble the particular single assay kit, the outer boxes from the storage locations by comparing the assay kit collection code on each outer box in order to verify they are associated with the particular single assay kit.

22.-29. (canceled)

30. A method of manufacturing an assay kit comprising:
inputting customer order information for a particular single assay kit into a database accessible by computer and retrieving from the database preloaded identification of reagents for the particular single assay kit;
displaying a location map for the particular assay kit indicating where in an insert a plurality of assay kit reagent containers comprising the reagents for the particular single assay are to be located, wherein the insert comprises an upper surface, a bottom surface, and a plurality of through holes extending from the upper surface of the insert to the bottom surface of the insert, and wherein each of the through holes is configured to hold a respective assay kit reagent container;
preparing a loaded insert by providing the insert and providing the plurality of identified assay kit reagent containers and disposing in accordance with the location map each of the assay kit reagent containers into a respective through hole of the plurality of through holes, wherein all or a subset of the assay kit reagent containers has a container bottom comprising a product code identifying the reagent within a particular container, and wherein the product code is readable by a human or a machine or both through the respective through hole at the bottom surface of the insert; and
inputting the product code on each container bottom comprising the product code within the loaded insert into the database and comparing the product code on each container with the location map to verify that the assay kit reagent containers in the loaded insert are loaded in accordance with the location map for the particular single assay kit.

31.-38. (canceled)

39. A package for an assay kit comprising:
a) an outer box; and
b) one or more removable trays,
i) wherein the outer box comprises a window and at least one of the trays comprises assay product information, and wherein the assay product information is viewable through the window; or
ii) wherein the at least one of the trays comprises an insert that comprises an upper surface, a bottom surface, and a plurality of through holes extending through the insert from the upper surface to the bottom surface, and wherein each of the plurality of through holes is shaped to individually hold a respective assay kit reagent container such that a product code located on all or a subset of the bottoms of the assay kit reagent containers is readable through the respective through hole at the bottom surface of the insert, and wherein the insert for at least one of the trays is removable from the tray, or comprises a portion that is removable from the tray, and wherein the removable insert or the removable portion is adapted to be placed on a scanner to read the product code; or
iii) both (i) and (ii).

40.-56. (canceled)
